(19) United States
(12) Patent Application Publication
(10) Pub. No.: US 2012/0088659 A1
(43) Pub. Date: Apr. 12, 2012

(54) Compositions and Methods for Delivery of Materials

(75) Inventors: David Taft, Atherton, CA (US); Steven Bitler, Menlo Park, CA (US); Qiang Zheng, Palo Alto, CA (US); Natarajan Balanchander, West Lafayette, IN (US); Damian Hadjuk, San Jose, CA (US)

(73) Assignee: Landec Corporation, Menlo Park, CA (US)

(21) Appl. No.: 13/376,191

(22) PCT Filed: Jun. 4, 2010
(86) PCT No.: PCT/US2010/037382

§ 371 Date: Dec. 5, 2011

Related U.S. Application Data
(60) Provisional application No. 61/217,772, filed on Jun. 4, 2009.

(51) Int. Cl. A61K 47/32; A01N 25/10; A61K 8/02; A01N 25/26; A61K 8/81; A61K 9/00; B82Y 30/00; B82Y 5/00

(52) U.S. Cl. 504/100; 514/772.6; 424/400; 977/700; 977/906; 977/902

(57) ABSTRACT

Compositions for the controlled delivery of active ingredients, for example, drugs, cosmetic materials, and agricultural materials. The compositions include a multiplicity of particles, each of the particles comprising (1) a microstructure comprising a semicrystalline polymer which has a peak melting temperature, Tp, of at least 20° C., and (2) the active ingredient. The microstructure comprises
(i) crystalline domains of the semicrystalline polymer, the crystalline domains being separate from each other, and
(ii) amorphous domains of the semicrystalline polymer, the amorphous domains providing a multiplicity of tortuous paths between the crystalline domains.

At least part of the active ingredient is present in the amorphous domains. One of many valuable uses for the compositions is to provide a coating on a seed, for example a coating containing one or more of an insecticide, a fungicide, a nutrient, a biotic, a growth regulator and an herbicide.

### CROSS-REFERENCE TO RELATED APPLICATIONS

This application claims priority from and the benefit of U.S. provisional Application No. 61/217,772, titled “Compositions and methods for delivery of materials” filed Jun. 4, 2009 (Docket 012 PRV),

This application is related to

(1) U.S. provisional Application No. 60/873,234, filed Dec. 5, 2006 (Docket 001 PRV),

(2) U.S. application Ser. No.11/999,415, filed Dec. 4, 2007 (Docket 001 US), which claims priority from and the benefit of (1), published as US 2008-026 11 9105

(3) U.S. provisional Application Nos. 61/005,400, filed Dec. 4, 2007 (Docket 003 PRV),

(4). International Patent Application PCT/US 2007/025032 (Docket 002 PCT), which was (i) filed Dec. 4, 2007, claiming priority from (1), and (ii) published as WO/2008/070165 on Jun. 12, 2008,

(5) International Patent Application PCT/US 2007/024909 (Docket 002 PCT), which was (i) filed Dec. 4, 2007, claiming priority from (1), and (ii) published as WO/2008/070118 on Jun. 12, 2008,

(6) U.S. provisional application 61/131,123, filed Jun. 4, 2008 (Docket 004 PRV),

(7) U.S. provisional application 61/131,716, filed Jun. 10, 2008 (Docket 004B PRV),

(8) U.S. application Ser. No 12/284,755, filed Sep. 25, 2008 (Docket 005 US), which is a continuation of U.S. application Ser. No. 11/999,415, and which claims priority from (3) and (6),

(9) U.S. application Ser. No. 12/287,520 filed Oct. 10, 2008 (Docket 006 US), which claims priority from (3), (6) and (7), published as US 2009-0209558

(10) International Patent Application PCT/US 2008/013335 (Docket 007 PCT), filed Dec. 3, 2008, claiming priority from (3), (6) and (7), and published as WO 2009073192,

(11) U.S. application Ser. No. 12/315,876, filed Dec. 4, 2008 (Docket 008 US), which claims priority from (3), (6) and (7),

(12) U.S. application Ser. No. 12/455,650 filed Jun. 4, 2009, (Docket 011 US), and entitled Compositions and Methods for Agriculture and Aquaculture, and which claims priority from (6) and (7), and which was published as 20100004124,

(13) U.S. application Ser. No. 12/455,628, filed Jun. 4, 2009, (Docket 010 US), and entitled Compositions and Methods for Personal Care, which claims priority from (6) and (7), and which was published as US 200900246155, and

(14) U.S. provisional application 61/268,329, filed Jun. 11: 2009 (Docket 009 PRV).

The entire disclosure of each of the applications and publications enumerated above is incorporated by reference herein for all purposes.

### FIELD OF THE INVENTION

This invention relates to polymeric systems for the delivery of useful materials.

### BACKGROUND

The related patent applications and publications enumerated above disclose the use of certain materials (referred to in U.S. application Ser. No. 12/315,876 as “CYC carriers”) for the delivery of useful materials, including but not limited to drugs which are useful in the treatment of human beings or other mammals, cosmetic materials and agricultural materials.

There are many known polymeric systems for the delivery of useful materials. Some of those systems make use of a side chain crystalline polymer. Reference may be made for example to the patent applications incorporated by reference above and to U.S. Pat. No. 4,830,855, U.S. Pat. No. 5,120,349, U.S. Pat. No. 5,129,180, U.S. Pat. No. 6,199,318, U.S. Pat. No. 6,224,793, U.S. Pat. No. 6,540,184, U.S. Pat. No. 6,858,634, U.S. Pat. No. 7,175,832, and U.S. Pat. No. 7,182,951; the entire disclosure of each of those patents is incorporated by reference herein for all purposes.

### SUMMARY OF THE INVENTION

This invention provides novel particulate compositions which are useful for the delivery of useful materials, methods of using those compositions, and methods of making those compositions. The materials to be delivered are referred to herein as “release materials” or “active ingredients”.

In a first aspect, the invention provides a composition which comprises a multiplicity of particles, each of the particles comprising (1) a CYC carrier as hereinafter defined, and (2) a release material which is an active ingredient as hereinafter defined, and which is associated with CYC carrier. The composition can also contain particles of a different kind. Preferably, each of the particles comprises a microstructure comprising

(i) first domains, preferably crystalline domains, which are separate from each other, and

(ii) second domains, preferably amorphous domains, which provide a multiplicity of tortuous paths between the first domains,

the release material (a) contacting the second domains (for example being at least partially dispersed in the second domains), (b) having a comparatively low rate of diffusion through the first domains, and (c) having a comparatively high rate of diffusion through the second domains. The first domains or the second domains or both the first domains and the second domains comprise the CYC carrier as hereinbefore defined.

In a second aspect, the invention provides a composition which comprises a multiplicity of particles, each of the particles comprising (1) a microstructure comprising a semicrystalline polymer which has a peak melting temperature, Tp, of at least 20° C., measured as hereinbefore defined, and (2) an active ingredient which is a release material as hereinbefore defined;

the microstructure comprising

(i) crystalline domains of the semicrystalline polymer, the crystalline domains being separate from each other, and

(ii) amorphous domains of the semicrystalline polymer, the amorphous domains providing a multiplicity of tortuous paths between the crystalline domains; and

at least part of the release material being present in the amorphous domains. The composition can also contain particles of a different kind. The crystalline polymer is preferably a CYSC polymer (a particular class of CYC polymer) as hereinafter defined, in which case the composition is an example of the compositions of the first aspect of the invention.

The microstructure which is present in the particles of the second aspect of the invention and which is preferably present in the particles of the first aspect of the invention preferably has a characteristic length scale of 5-1,000 nm, e.g. 10-500 nm. The characteristic length scale referred to in this specification is measured by small-angle X-ray scattering and is manifested as a maximum in the corresponding region of reciprocal space for small-angle.

X-ray scattering data collected from the composition.

In a third aspect, this invention provides a method of making a particulate composition according to the first aspect of the invention, the method comprising

(A) dispersing the release material in the CYC carrier while the release material is a solid and the CYC carrier is a liquid, the dispersing being carried out in the absence of any solvent for the release material; and

(B) converting the dispersion prepared in step (A) into the particles.

In a fourth aspect, this invention provides a method of making a particulate composition according to the first aspect of the invention, the method comprising

(A) dispersing the release material in the CYC carrier while the release material is a liquid and the CYC carrier is a liquid, the dispersing preferably being carried out in the absence of any solvent for the release material; and

(B) converting the dispersion prepared in step (A) into the particles.

In a fifth aspect, this invention provides a method of making a particulate composition according to the second aspect of the invention, the method comprising

(A) dispersing the release material in the semicrystalline polymer while the release material is a solid and the semicrystalline polymer is at a temperature above Tp, preferably at least (Tp+25) ° C., the dispersing preferably being carried out in the absence of any solvent for the release material; and

(B) converting the dispersion prepared in step (A) into the particles. In a sixth aspect, this invention provides a method of making a particulate composition according to the second aspect of the invention, the method comprising.

(A) dispersing the release material in the semicrystalline polymer while the release material is a liquid and the semicrystalline polymer is at a temperature above Tp, preferably at least (Tp+25) ° C., the dispersing preferably being carried out in the absence of any solvent for the release material; and (B) converting the dispersion prepared in step (A) into the particles.

In further aspects, this invention provides
(7) Methods of releasing release materials from compositions as defined above in order to affect (e.g. improve, preserve, identify or damage) a target site, e.g. to affect the health or appearance of an organism, or to assist in the diagnosis of an organism, the method comprising exposing the composition to a liquid which is soluble in the amorphous domains, while the composition is at a temperature below Tp and preferably at a temperature above the glass transition temperature, Tg, of the polymer.
(8) Methods of affecting a target site which comprise administering a composition as defined above to a target site.
(9) Devices for administering a release material, the device comprising a composition as defined above.
(10) The use of a release composition or release device as defined above.

### BRIEF DESCRIPTION OF THE DRAWINGS

The invention is illustrated in the accompanying drawings in which the Figures summarize results obtained in the Examples, as further described below. In each of the Figures, the vertical axis shows the total percentage release (“cumulative release”) of the release material, the horizontal axis shows the time in hours, and the different curves are identified by reference to the Example # marked on the Figure.

FIG. 1: The release profiles were run at a constant 30 C under infinite dilution condition in water. % of Imidacloprid released is plotted as a function of time (hrs).

FIG. 2: The release profiles were run at a constant 30 C under infinite dilution condition in water for 150 hrs, moved to 40 C till 420 hrs and then at 70 C. % of lmidacloprid released is plotted as a function of time (hrs).

FIG. 3: The release profiles were run at a constant 30 C under infinite dilution condition in water. % of Imidacloprid released is plotted as a function of time (hrs).

FIG. 4: The release profiles were run at a constant 30 C under infinite dilution condition in water. % of Imidacloprid released is plotted as a function of time (hrs).

### DETAILED DESCRIPTION OF THE INVENTION

In this specification:—
(1) Reference is made to particular features of the invention (including for example components, ingredients, elements, devices, apparatus, systems, groups, ranges, method steps, test results, etc). It is to be understood that the disclosure of the invention in this specification includes all possible combinations of such particular features. For example, where a particular feature is disclosed in the context of a particular embodiment or claim, that feature can also be used, to the extent appropriate, in the context of other particular embodiments and claims, and in the invention generally.
(2) The singular forms “a”, “an”, and “the” include plural referents unless the context dictates otherwise. Thus, for example, a reference to “a part” includes a plurality of such parts.
(3) The term “comprises” and grammatical equivalents thereof are used to mean that, in addition to the features specifically identified, other features are optionally present. For example a formulation which comprises a CYC carrier and a drug can contain a single CYC carrier and a single drug, or two or more CYC carriers and/or two or more drugs, and optionally contains one or more other ingredients which are not CYC carriers, for example other ingredients as disclosed herein.
(4) The term “consisting essentially of” and grammatical equivalents thereof are used to mean that, in addition to the features specifically identified, other features may be present which do not materially alter the disclosed and/or claimed invention.
(5) The term “at least” followed by a number is used to denote the start of a range beginning with that number (which may be a range having an upper limit or no upper limit, depending on the variable being defined). For example “at least 1” means 1 or more than 1, and “at least 80%” means 80% or more than 80%.
(6) The term “at most” followed by a number is used to denote the end of a range ending with that number (which may be a range having 1 or 0 as its lower limit, or a range having no lower limit, depending upon the variable being defined). For example, “at most 4” means 4 or less than 4, and “at most 40%” means 40% or less than 40%.
(7) A range written as “(a first number) to (a second number)” or “(a first number)-(a second number)” means a range whose lower limit is, the first number and whose upper limit is the second number. For example, “from 8 to 20 carbon atoms” or “8-20 carbon atoms” means a range whose lower limit is 8 carbon atoms, and whose upper limit is 20 carbon atoms.
(8) The terms “plural”, “multiple”, “plurality” and “multiplicity” are used herein to denote two or more than two features.
(9) When a method is described as comprising two or more defined steps, the defined steps can be carried out in any order or simultaneously (except where the context excludes that possibility), and the method can optionally include one or more other steps which are carried out before any of the defined steps, between two of the defined steps, or after all the defined steps (except where the context excludes that possibility).
(10) When reference is made to two or more features, this includes the possibility that the two or more features are replaced by a lesser number or greater number of features providing the same function (except where the context excludes that possibility).
(11) The numbers given should be construed with the latitude appropriate to their context and expression; for example, each number is subject to variation which depends on the accuracy with which it can be measured by methods conventionally used by those skilled in the art.
(12) Parts, ratios and percentages are by weight, except where otherwise noted.
(13) Temperatures are in degrees Centigrade (° C.).
(14) Molecular weights of polymers are in Daltons; are number average molecular weights (Mn) unless stated to be weight average molecular weights (Mw); and are measured by gel permeation chromatography (GPC) with a light scattering detection method, for example using a DAWN DSP laser photometer from Wyatt Technology, unless stated to be measured using GPC against a polystyrene standard.
(15) The terms “melting point” (often abbreviated to Tp), “onset of melting temperature” (often abbreviated to To) and “heat of fusion” (which is a measure of the crystallinity of the polymer, is expressed in J/g and is often abbreviated to ΔH) are well known to polymer technologists and refer to quantities determined using a differential scanning calorimeter (hereinafter DSC), e.g. a Q 100 DSC from TA Instruments, at a rate of temperature change of 10° C./min, e.g. from −10 to 150° C., unless otherwise noted. Tp is the peak melting temperature, To is the temperature at the intersection of the baseline of the DSC peak and the onset line, the onset line being defined as the tangent to the steepest part of the DSC curve below Tp, and ΔH is the heat of fusion associated with the endotherm or exotherm as calculated by the DSC and is reported in J/g. Unless otherwise noted, the values of Tp, To and ΔH are measured on the second heat cycle.
(16) Bulk viscosities are given in centipoise and are measured using a Brookfield LVT viscometer with an electronically thermostat controlled thermal heater, controlled for example to about 95° C., and small sample adapter using spindles 4 and 7, or, in the Examples below, spindle 18.
(17) Solubility parameters are calculated using the method described in D. W. van

Krevelen, “Properties of Polymers” Elsevier, 1997, p. 200-214 especially p. 214 and reported in J^{1/2}/cm^{3/2}.
(18) The term “associated” and grammatical variations thereof include any type of interaction, including chemical bonds (for example, covalent, ionic and hydrogen bonds) and/or Van der Waals forces, and/or polar and non-polar interactions through other physical constraints provided by molecular structure, and interactions through physical mixing.
(19) The term “pharmaceutical formulation” means a composition which (i) is suitable for administration to a human being or other mammal or which can be treated, e.g. sterilized, to make it suitable for such administration, and (ii) comprises at least one drug and at least one CYC carrier. The term “drug” means a material which is biologically active in a human being or other mammal, locally and/or systemically.
(20) The term “therapeutically effective amount” or “therapeutically effective dosage” means an amount of a drug which results in a desired therapeutic effect.
(21) The term “agricultural” refers to compositions and methods which are useful in the treatment of plants, seeds and soil. The term “aquacultural” refers to compositions and methods which are useful in the treatment of fresh water or salt water organisms.
(22) The term “Personal Care” refers to compositions and methods which are useful in the treatment of the human body (including human hair) primarily or exclusively for non-medical purposes, for example for cosmetic or hygienic purposes.
(23) The term “organism” includes, but is not limited to, human beings and other mammals, living tissue which is not part of a mammal, freshwater organisms, saltwater organisms, plants, seeds, and soil which contains living organisms. The invention is useful, for example, for delivering drugs to human beings and other mammals; for delivering biocides and/or fertilizers to plants, seeds and soil; for delivering cosmetic ingredients to human beings; and for use as components in cosmetic and other formulations to provide benefits such as thickening and control of rheological properties.
(24) The term “therapeutically effective amount” means an amount of a drug which produces a desired therapeutic effect.
(25) The term “diagnostic agent” means any chemical moiety that can be used for diagnosis or in a diagnostic test. For example, diagnostic agents include imaging agents containing radioisotopes, contrasting agents containing for example iodine, enzymes, fluorescent substances and the like.
(26) The term “treatment” means administration of a composition to a site, e.g. the administration of a pharmaceutical formulation to an individual in order to alter a physiological state, whether or not the process includes a curative element.
(27) “Controlled release” of a drug or other bioactive material means release of the material in a pre-determined or adjustable way such that the amount or rate or timing of release is pre-set or is altered in a desired way.
(28) The terms “controlled release device”, “controlled release dosage form” and similar terms mean any formulation or device wherein the release rate (e.g., rate of timing of release) of a drug or other desired substance contained therein is controlled by the device or dosage form itself and/or by the environment of use. Controlled drug delivery includes delivery of an amount of drug to a particular target site at a particular time, for example delivery of a bolus of drug to a tumor site.
(29) “Sustained release” of a drug or other material means release over an extended period of time, for example minutes, hours or days, such that less than all the bioactive material is released initially. A sustained release rate may provide, for example, a release of a specified amount of a drug from a pharmaceutical formulation, over a certain time period, under physiological conditions or in an in vitro test. With particular reference to release profiles of drugs/active agents, the disclosure of WO2009/073192 is incorporated by reference in its entirety.
(30) “Bolus” release means release of a large dose, for example substantially all of a drug at one time or over a short period of time. Bolus release can be preceded or followed by sustained release.
(31) The term “burst effect” is used, often in the context of drug delivery, to mean release of a bioactive material from a composition in an amount or at a rate which is higher than is desired (typically, in drug delivery, higher than the therapeutic window). A burst is generally followed by a rapidly decreasing rate of release. A burst effect may be defined as the release of more than a defined threshold proportion of the bioactive material over a defined time under defined conditions.
(32) The term “functionalized”, as applied to a chemical compound, including a polymer, means that the compound has been treated so that it contains a functional moiety (i.e. a moiety which will undergo a further desired chemical reaction) which was not present on the compound before the treatment, or so that the polarity of the compound is changed, as evidenced, for example, by a change in the solubility parameter.
(33) The term “alkyl” includes alkyl moieties which are straight chain alkyl moieties, branched chain alkyl moieties, cycloalkyl moieties, and moieties which consist essentially of two or more of straight chain alkyl, branched chain alkyl and cycloalkyl moieties.
(34) The term “bioerodable” (sometimes alternatively “biodegradable”) as applied to a CYC carrier or to a release composition means that the carrier or composition, when placed in the human body, is eliminated from the human body, the carrier being eliminated without change or as one or more lower molecular weight products resulting from the degradation of the carrier in the human body. The elimination may take place relatively rapidly, e.g. in a period of up to 10 weeks, but is more often longer, for example over a period of up to 1 year or longer, e.g. up to 3 years. Carriers that are not bioerodable may leave the body by voiding if part of an oral formulation, or by explantation if part of an implanted formulation.
(35) Some of the structural formulas given below for CYC polymers show the repeating units in the general form

-(-unit#1-)ₓ-(-unit#1-)_{y}-

This representation is used to denote polymers in which the different repeating units are distributed randomly and/or are distributed in blocks containing only one of the repeating units. Thus, the polymers represented by these formulas can be either random copolymers or block copolymers.

This specification incorporates by reference all documents referred to herein and all documents filed concurrently with this specification or filed previously in connection with this application, including but not limited to such documents which are open to public inspection with this specification.

### Definition of CYC Carrier

The term CYC carrier is used herein to include CYC polymers, CYC compounds, CYC assemblies and mono-Cy CYC compounds, each of which is defined below. Particular classes of CYSC polymers are CYSC polymers, ECC polymers and SSP polymers, which are also defined below. A particular class of CYC compounds are “supplementary CYC compounds”, which are also defined below. Where the disclosure below refers to one of the CYC carriers, a component of one of the CYC carriers, a characteristic of one of CYC carriers, or to a composition or method making use of one of the CYC carriers, that disclosure is also applicable to other CYC carriers, unless the context makes this impossible.

The term “CYC polymer” is defined herein as a polymer which:
(A) comprises polymeric molecules having a backbone and comprising at least one moiety which

(i) has the formula -b-Cy, and (ii) either

(A) forms part of a repeating unit of the backbone, the repeating unit having formula (1) below

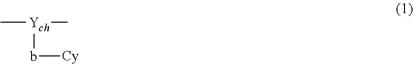

where Y_{ch} is a moiety forming part of the backbone,
b is a bond or moiety which links the Cy moiety to Y_{ch}, and
Cy is a moiety which is associated with other moieties (which may also be Cy moieties) to provide the CYC polymer with crystallinity;

or

(B) forms part of a terminal unit of the backbone, the terminal unit having formula (2) below

-Yₜₑᵣₘ-b-Cy (2)

where Yₜₑᵣₘ is a moiety at the end of the backbone, and
b and Cy are as defined in formula (1); and
(B) has a crystalline melting temperature (hereinafter abbreviated to Tp) of at least 0° C. and a heat of fusion (hereinafter abbreviated to ΔH) of at least 3 J/g which result from association of the Cy moieties. In this definition, and throughout this specification, Tp and ΔH are measured on a differential scanning calorimeter (DSC) as hereinafter described.

In many CYC polymers, the backbone of the polymeric molecules comprises repeating units having formula (3) below

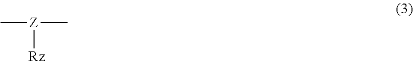

where Z is a moiety forming part of the backbone and Rz represents a moiety which does not comprise a Cy moiety. Many useful CYC polymers have an amphiphilic character, with the Cy moieties providing hydrophobic characteristics and the Z(Rz) moieties providing hydrophilic characteristics.

The term “CYSC polymer” is used herein to denote a CYC polymer in which at least a majority by weight, preferably at least 90% by weight, particularly substantially all, of the Cy moieties are present in repeating units of formula (1). Thus, a CYSC polymer always contains repeating units of formula (1), and optionally contains terminal units of formula (2) and repeating units of formula (3). Patents and other publications relating to CYSC polymers include J. Poly. Sci. 60, 19 (1962); J. Poly. Sci, (Polymer Chemistry) 7, 3053 (1969), 9, 1835, 3349, 3351, 3367, 10, 1657, 3347, 18, 2197, 19, 1871; J. Poly. Sci, Poly-Physics Ed 18 2197 (1980); J. Poly. Sci, Macromol. Rev, 8, 117 (1974); Macromolecules 12, 94 (1979), 13, 12, 15, 18, 2141, 19, 611; JACS 75, 3326 (1953), 76; 6280; Polymer J 17, 991 (1985); and Poly. Sci USSR 21, 241 (1979); U.S. Pat. Nos. 4,830,855, 5,120,349, 5,129,180, 5,156,911, 5,254,354, 5,387,450, 5,412,035, 5,469,867, 5,665,822, 5,752,926, 5,783,302, 6,013,293, 6,060,540, 6,199,318, 6,210,724, 6,224,793, 6,255,367, 6,376,032, 6,492,462, 6,540,984, 6,548,132, 6,831,116, 6,989,417, and 7,101,928; and US Patent Application Publications Nos. 2001/0018484, 2002/0090425 and 2002/0127305. The entire disclosure of each of those publications, patents and patent publications is incorporated herein by reference for all purposes.

The term “ECC polymer” is used herein to denote a CYC polymer in which at least a majority by weight, preferably at least 90% by weight, particularly substantially all, of the Cy moieties are present in terminal units of formula (2). Thus, an ECC polymer always contains terminal units of formula (2) and repeating units of formula (3), and optionally contains repeating units of formula (1). The term SSP polymer is defined herein as a polymer which

(1) has a crystalline melting temperature, Tp, of at least 25° C., e.g. 27-100° C., and a ΔH of at least 5 J/g; and

(2) comprises polymeric molecules having a backbone which comprises
(a) repeating units which do not contain hydrophilic moieties, and have the formula (1) below,

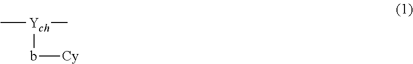

where Y_{ch} is a moiety forming part of the backbone,
b is a bond or a moiety linking the Cy moiety to Y_{ch}, and
Cy is a moiety is associated with other Cy moieties to provide the SSP polymer with crystallinity;
(b) repeating units which have the formula (2zphil) below,

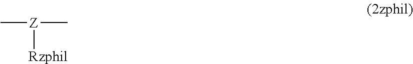

where Z is a moiety forming part of the backbone, and
Rzphil comprises a hydrophilic moiety; the molar ratio of the units of formula (2zphil) to the units of formula (1) being at least 2.5:1.

The term “CYC compound” is defined herein as a non-polymeric compound which

(A) has the formula

Q(-b-Cy)_{q} (4)

wherein q is least 2, e.g. 3-8,
Q is a moiety having a valence of at least q,
b is a bond or a moiety linking the Cy moiety to the Q moiety, and
Cy is as defined in formula (1), and

(B) has a crystalline melting temperature, Tp, of at least 0° C. and a ΔH of at least 3 J/g which results from association of the Cy moieties.

The term “mono-Cy CYC carrier” is defined herein as a non-polymeric compound which:

(A) has the formula.

Q-Cy

wherein Cy is as defined in formula (1) above, and
Q is a moiety which (i) is free of Cy moieties and (ii) contains at least one functional moiety, for example a hydrophilic moiety, thus resulting in a compound which is an amphiphile, or a polar group which can associate with a suitable release material, or a functional group which can form a covalent link with a suitable release material; and

(B) has a crystalline melting temperature, Tp, of least 0° C., e.g. at least 25° C., e.g. 27-100° C., and a ΔH of at least 3 J/g.

The term “supplementary CYC carriers” is used herein to denote a CYC carrier, for example an ECC-PLGA polymer, which has been modified by covalent bonding to a silicon-containing moiety, for example a hydroxy poly-alkyl siloxane.

The term “CYC assembly” is defined herein as an assembly of (i) a polymer which is a CYC polymer as defined above except that the polymer does not necessarily have a Tp of at least 0° C. and a ΔH of at least 4 J/g, and (ii) a compound which contains a Cy moiety and which is intimately mixed with the polymer but is not covalently linked to the polymer, the assembly having a crystalline melting temperature, Tp, of at least 0° C. and a ΔH of at least 3 J/g which results from association of the Cy moieties.

The moiety -b-Cy is also referred to in this specification as an -Rc moiety, i.e. Rc is synonymous with b-Cy.

Additional information about CYC carriers is to be found in the documents incorporated by reference herein, in particular pages 15-78 of PCT/US 2008/013335, published as WO 2009/073192.

### The Ingredients of the Particles

### (A) Release Materials

A wide variety of active ingredients can be used as release materials in this invention. The active ingredients include drugs, cosmetic materials, agricultural materials, catalysts, and inhibitors.

The term “drug” is used herein to denote a material which is biologically active in a human being or other mammal, locally and/or systemically, including any chemical moiety that can be used for diagnosis or in a diagnostic test. The term “drug” includes food additives, including nutrients, used in food supplements for animal or human consumption, and antimicrobials (i.e. materials that attack bacteria and other microbes). Examples of drugs are disclosed in the Merck Index, the Physicians Desk Reference, and in column 11, line 16, to column 12, line 58, of U.S. Pat. No. 6,297,337, and in paragraph 0045 of U.S. 2003/0224974, the entire disclosures of which are incorporated by reference herein for all purposes.

The term “cosmetic material” is used herein to denote a material which is not a “drug” as defined above and which, at the time of delivery or after delivery to the human body, changes (generally improves or preserves) the appearance of, and/or hygiene of, and/or smell associated with, the human body. The cosmetic materials can be delivered in combination with materials which are “drugs” as defined above. The personal care sites to which the cosmetic materials can be delivered (which are also referred to herein as “target sites”) can be any part of the human body (including, but not limited to, skin, hair and nails). Compositions for personal care, e.g. hair care, skin care, sun care, color care and body care, have been disclosed for example in US Patent and Patent Publications U.S. Pat. Nos. 6,540,984, 6,989,417, 7,101,832 and 7,175,832 (Landec Corporation); U.S. Pat. No. 5,736,125 (National Starch Corporation); U.S. Pat. Nos. 5,622,694, 5,662,892, 5,916,547, 5,919,439, 6,074,628 and 6,113,883 (Procter and Gamble Corporation); and U.S. Pat. Nos. 6,503,494, 6,572,869, 6,361,781, 6,569,409, 6,464,969, 6,565,839, 7,335,348, 6,789,550, 6,811,770, 6,949,504, 7,129,276, 7,255,870, 6,946,518, 7,090,420, 7,083,347, 2002041857, 2002127251, 2003039621, 2004005279, 2005188474, 2005191262, 2005172421, 2005191258, 2004180021, 2004191200, 2004228890, 2005031656, 2005013838, 2005142082, 2005123493, 2005008667, 2005031565, 2005169949, 2005169865, 2005261159, 2005175570, 2005180936, 2006078519, 2005287093, 2005287183, 2005287100, 2005287101, 2006078520, 2006130248, 2006233732, 2006216257, 2006292095, 2006263438, 2007134192, 2005137117 and 2003039671 (L'Oreal). The entire disclosure of each of those patents and patent publications is incorporated herein by reference for all purposes, including their disclosure of “cosmetic materials”.

The term “agricultural material” is used herein to denote a material which is not a “drug” as defined above and which, at the time of delivery or after delivery to the agricultural or aquacultural site, produce a desired effect at the agricultural or aquacultural site, for example on live vegetable matter (including seeds), or on live fish, pests, for example insects and fungus, or on the environment surrounding live vegetable matter or live fish, or on soil which surrounds a seed or in which a plant is growing. In many cases, the agricultural material is bioactive, for example is a biocide (e.g. a pesticide such as a fungicide, a mildewicide or an insecticide), or a fertilizer, plant hormone or enzyme. In some embodiments of the invention, a composition of the invention is coated onto a seed, or mixed with soil, or applied to the leaves of a growing plant. Agricultural materials can also be used in other contexts, for example mildewcides are widely used to control the growth of mildew and fungi in buildings and other sites. Specific examples of bioactive materials include Thiram, Fludioxanil, Captan, Rival, and Apron and insecticides such as imidacloprid, chlothianidin, dinotefuran and thiomethoxam. The invention is particularly useful for the treatment of seeds, for example to provide a seed coating which contains one or more of an insecticide, a fungicide, nutrient, biotic, growth regulator and herbicide. The insecticide can for example be a nicotinic acetyl choline receptor agonist (IRAC 4A) or neonicotinoid, e.g. clothianidin (Poncho), imidacloprid (Gaucho), and thiamethoxam (Cruiser); or a chloride channel activator (IRAC 6) e.g. abamectin (Avicta) or friponil (Regent). The fungicide can for example be a sterol synthesis (FRAC 3), e.g. a triazole such as ipconazole (Vortex) or difenoconazole (Dividend); a nucleic acid synthesis (FRAC 4), e.g. an acylalanine such as metalaxyl (Allegiance) or mafaenoxam (Apron); a respiration (FRAC 11), e.g. a methoxyacrylate such as azoxystrobin (Dynasty), a methoxycarbamate such as pryaclostrobin (Stamina F 500); or an oximino acetate such as trifloxystrobin (Trilex); a signaling (FRAC 12), such as fludioxonil (Maxim); a multi-site contact activity (FRAC M) such as a dithiocarbamate ((Mancozeb, Manab, Thiram, Ziram) or a phthalimide (Captan). The plant growth regulator can for example be a systemic acquired resistance (SAR), such as E. Coli K12 or Harpin ab protein. The herbicide can for example be a lipid synthesis inhibitor which is an acetyl

CoA carboxylase of the aryloxyphenoxy propionate family, e.g. fenoxyprop (a component of Fusion), fluazifop (Fusilade DX) or quizalofop (Assure II, Targa) or of the cyclohexanedione family, e.g. clethodiim (Select, Arrow) or sethoxydim (Poast, Poast Plus); or an EPSP synthesis inhibitor, e.g. glypophoshate (Roundup, Touchdown); and amino acid synthesis inhibitor, e.g. an ALS inhibitor of the sulfonylurea family, e.g. chloroimuron (Classic), foramsulfuron (Option), halosulfuron (Permit), iodosulfuron (Autumn) or nicosulfuron (Accent) or of the imidazolinone family, e.g. imazaquin (Raptor), imaxaquin (Scepter). All imazethapyr (Pursuit), or of the triazoloyrimidine family, e.g.flumetsulam (Python) or cloransulam (First Rate); or a growth regulator (a synthetic auxin) of the phenoxy family, e.g. 2,4-D (Weedone and others), or of the benzoic acid family, e.g. dicamba (Banval, Clarity), or of the carboxylic acid family, e.g. clopyralid (Stinger) or fluroxypyr (Starane), or of the semicarbazone family, e.g. diflufenzopyr (a component of Distinct); a photo synthesis inhibitor of the triazine family (e.g. atrazine (AAtrex and others), simazine (Princep), or of the triazinone family, e.g. metribuzin (Sencor), or of the nitrile family, e.g. bromoxynil (Buctril), or of the benzothiadiazole family, e.g. bentazon (Basagran) or of the urea family, e.g. ilinuron (Lorox, Linex); a nitrogen metabolism, e.g. glufosinate (Liberty); a pigment inhibitor of the isoxazolidinone family, e.g. clomazone (Command), or of the isoxazole family, e.g. isoxaflutole (Balance Pro), or of the triketone family, e.g. mesotrione (Callisto, Laudis, Impact); or a cell membrane disruptor of the diphenylether family, e.g. acifluoren (UltraBlazer), fomesafen (FlexStar, Reflex), lactofen (Cobra, Phoenix), or of the N-phenylphthalimide family, e.g. flumiclorac (Resource), flumioxazin (Valor SX), or of the aryl triazinone family, e.g. sulfentrazone (Spartan), carfentrazone (Aim), or of the bipyridilium family, e.g. paraquat (Gramoxone Inteon); or a seedling root growth inhibitor of the dinitroaniline family, e.g. ethafluralin (Sonalin), pendimethalin (Prowl and others), and trifluralin (Treflan and others); or a seedling shoot growth inhibitor of the thiocarbamate family, e.g. butylate (Sutan+), EPTC (Eradicane), or of the chloroacetamide family, e.g. acetochlor (Harness, TopNotch, Degree),alachlor, e.g. Intro, MicroTech), metalochlor (Dual II, Magnum and others), dimethanamid (Outlook, Propel), or of the oxyacetamide family, e.g. flufenacet family (Define).

For additional disclosure of active ingredients, reference may be made to the documents incorporated by reference herein.

In the particles having the preferred microstructure, the active ingredient is preferentially present in the amorphous domains. The crystalline domains may be substantially free of active ingredient. The concentration of active ingredient in the amorphous domains is for example at least 10 times the concentration in the crystalline domains. The active ingredient may also be present in separate domains consisting essentially of the active ingredient itself or comprising the active ingredient dispersed or dissolved in an additional ingredient, for example an amorphous polymer.

### (B) Polymers

The particles preferably include a semicrystalline polymer, since such polymers can be processed to provide a microstructure comprising first and second domains as described above. The semicrystalline polymer, preferably has one or more of the following characteristics.

(1) It is a CYSC polymer, for example a polyacrylate, as defined above.

(2) It has a heat of fusion of at least 20, preferably at least 30, particularly at least 40, J/g, measured as hereinbefore before defined.

(3) It has a peak melting temperature, Tp, and an onset of melting temperature, To, such that (Tp−To) is less than Tp^{0.7}, preferably less than Tp^{0.6}, particularly less than 10° C., measured as hereinbefore defined.

(4) It has a peak melting temperature, Tp, of at least 30° C., e.g. at least 35° C., for example 35-80° C., or 50-65° C., or at least 50° C., e.g. 15-85° C., measured as hereinbefore defined.

(5) It has a number average molecular weight of 2,000-60,000, preferably 5000-50,000, or 5000-30,000, particularly 10,000-20,000, or 15,000-25,000, and/or a weight average molecular weight of 2000-100,000, e.g. 10,000-100,000, preferably 15,000-25,000.

(6) It has a melt viscosity (measured as hereinafter described) less than 1000 cps, e.g. less than 500 cps.

(7) It contains repeating units containing functional groups, preferably hydrophilic groups, for example units derived from monomers containing one or more of oxygen-, nitrogen- or silicon-containing groups, e.g. carboxyl, hydroxyl, amino, substituted amino, amido or polyoxyethylene groups, the polymer containing for example at least 5%, at least 10%, at least 15%, at least 30%, or at least 40%, in some cases 30-50%, by weight of such units.

The CYSC polymers are preferred, particularly when they have one or more, preferably all, of characteristics (1)-(6), because sufficiently rapid cooling of a homogeneous mixture of such a CYSC polymer and an active ingredient results in the preferred microstructure defined above. CYSC polymers having characteristic (7) are preferred, particularly when release of the active ingredient takes place in an aqueous environment, because they encourage access of water to the amorphous domains, and, therefore, release of the active ingredient.

### (C) Additional Ingredients of the Particles

The particles can optionally include one or more additional ingredients, the ingredient having one or more of the following characteristics:—

(a) It decreases the glass transition temperature, Tg, of the polymer.

(b) It increases the Tg of the polymer.

(c) It increases the Tp of the polymer.

(d) It increases the solubility of the active ingredient in the polymer.

(e) It modifies the active ingredient or the polymer to improve compatibility of the active ingredient and the amorphous domains of the polymer.

(f) It modifies the surface properties of the active ingredient or the polymer to improve compatibility of the active ingredient and the crystalline domains of the polymer.

(g) it provides additional crystalline domains.

(h) it provides additional amorphous domains.

These additional ingredients influence the way in which the active ingredient is released from the particles, and/or the stability of the microstructure. In choosing an additional ingredient, care should be taken that it does not destabilize the microstructure, for example by facilitating changes in active ingredient domain size during storage, and/or reducing the tortuosity of the amorphous domains. In some embodiments, an additive which increases the Tg of the polymer is useful because it stabilizes the amorphous domains. In other embodiments, an additive which decreases the Tg of the polymer is useful because it makes the amorphous domains more easily accessible to water or other medium which displaces the active ingredient when delivery of the active ingredient is desired.

The additional ingredient can be associated with the polymer and/or the active ingredient in any way, for example by interaction between similar chemical groups or by chemical reaction. In some embodiments, the particles are preferably free from any ingredient which is a solvent for either the active ingredient or the semicrystalline polymer.

Specific examples of additional ingredients include the following.

(1) Water, an organic liquid, a plasticizer or a low melting solid preferably having a melting point less than (Tp−10)° C.

(2) Compounds (including monomers and high and low molecular weight polymers) containing groups which can associate with the groups of the semicrystalline polymer which cause its crystallinity, for example hydrophobic groups, e.g. long chain alkyl groups, which associate with side chains of a CYSC polymer. Examples of such compounds include
(a) compounds containing straight chain alkyl groups containing at least 10, preferably at least 16, carbon atoms, e.g. an amorphous or crystalline fatty alcohol, or a fatty acid ester alcohol of a polyhydric alcohol which has been an ethoxylated to attach a polyoxyethylene chain containing a plurality, e.g. 2-20, preferably 2-6, oxyethylene units;
(b) a polymer obtained by polymerizing one or more monomers containing Cy groups and/or functional groups that provide the polymer with hydrophilic characteristics, for example a polymer obtained by polymerizing a Cy-containing monomer which is free of functional groups with up to 30%, preferably less than 20%, or more preferably less than 12%, by weight of a vinyl carboxylic acid, e.g. methacrylic acid or acrylic acid;
(c) an ECC polymer, for example a crystalline polymer obtained by endcapping PLGA, polycarbonate, polyethylene glycol, or a mixture of polyethylene glycol and poly propylene glycol.

(3) A crystalline alcohol having a melting point less than Tp, e.g. neopentyl glycol, t-butyl alcohol, or cyclohexane dimethanol.

(4) An amorphous polymer, e.g., a polyethylene glycol of molecular weight less than 1000, a polymethyl acrylate, or a polymethyl methacrylate.

(5) A main chain crystalline polymer having a low to medium molecular weight, e.g. 1000-20,000, for example a polyethylene glycol e.g. of molecular weight greater than 1500, a copolymer of ethically glycol and propylene glycol e.g. a molecular weight greater than 1500, or polycaprolactone, e.g. of molecular weight greater than 10,000.

The particles can also contain conventional ingredients such as fillers (e.g. calcium carbonate, marble dust, titanium dioxide and talc), surface active agents, additives to improve drying characteristics, and colorants.

### Particles

The particles can for example have an average particle size of 0.5-250μ, e.g. 0.5-150, 10-150, 20-150, 20-70, or 0.5-25μ. Particle sizes given in this specification are measured using a Horiba LA-910 Laser Scattering Particle Size Distribution Analyzer.

### Methods For Preparing the Particles

Known methods of preparing microparticles (having, however, different characteristics and/or different ingredients) can be used to prepare the compositions of the invention. Such conventional methods make use of a solvent for the polymer or for the active ingredient or for both in the preparation of a mixture of the polymer and the active ingredient. However, the compositions of the invention are preferably prepared by the methods of the third, fourth, fifth and sixth aspects of the invention, which do not make use of such a solvent. The use of solvents is a practical disadvantage during the preparation of particles. Furthermore, presence of residual solvent in the particles can be a significant disadvantage, for example, can have phytotoxic effects on seeds coated with the compositions, thus lowering the germination rate and adversely affecting the yield of plants.

In step (B) of these preferred methods, the dispersion prepared in step (A) is converted into the particles. Step (B) is preferably carried out under conditions such that the particles have the preferred microstructure. Preferably, in step (B), the dispersion is cooled rapidly, so that crystallization of the crystalline portion of the semicrystalline polymer results in the formation of very small separate crystalline domains. Most, if not all, of the active ingredient is excluded from the crystalline domains, and thus remains dispersed in the amorphous domains, or forms separate domains consisting essentially of the active ingredient. CYSC polymers are preferred, particularly when they have the preferred characteristics set out above, because they can be easily mixed with the active ingredient (if desired, at relatively low temperatures) and crystallize very rapidly when the dispersion is cooled. Polymers having higher values of (Tp−To) crystallize more slowly and require much greater amounts of energy input in order to form similar microstructures.

Specific methods of preparation include the following.
Method A. A homogeneous dispersion of the active ingredient in the polymer is produced by homogenizing a mixture of the polymer and the active ingredient at a temperature above the Tp of the polymer and below the melting point of the active ingredient. The homogeneous dispersion is then spun into particles on a spinning disk apparatus. Such apparatus is well known, and is described, for example, in U.S. Pat. No. 7,261,529, the entire disclosure of which is incorporated by reference herein. The particle size can be controlled by varying process conditions such as the flow rate, disk speed and temperature. The flow rate, is preferably at least 0.5-10 g/sec
Method B. The procedure of method A is used except that the homogenization is carried out at a temperature above the Tp of the polymer and above the melting point of the active ingredient.
Method C. A homogeneous dispersion of the active ingredient in the polymer is produced by homogenizing a mixture of the polymer and the active ingredient at a temperature above the Tp of the polymer and below the melting point of the active ingredient. The homogeneous dispersion is cooled to well below Tp, either gradually while mixing, or rapidly, e.g. in an ice-water bath. The resulting solid is ground to the desired particle size, for example by a standard grinding method or in a jet mill. This method may produce particles having a relatively high concentration of the active ingredient on the surface of the particles.
Method D. The procedure of method C is used except that the homogenization is carried out at a temperature above the Tp of the polymer and above the melting point of the active ingredient.

In each of these methods, the mixture that is homogenized preferably does not include a solvent for either the polymer or the active ingredient. However, the mixture can include one or more additional ingredients. The additional ingredient can be, for example, (1) a nucleating agent, which promotes rapid crystallization of the semicrystalline polymer into small crystalline domains, separated by amorphous domains, or (2) an additional polymer, which may be above or below its melting point during the homogenization, but which becomes a solid in the finished panicles. The additional polymer may or may not be a crystalline polymer. Other optional additional ingredients include the additives described above.

In each of these methods, the particles can be subject to a further treatment, which changes the shape or some other characteristic of the particle, for example a treatment in which the particles are heated to a temperature slightly above the Tp of the semicrystalline polymer, for example in a fluid bed. When the particles have been produced by grinding, such treatment can reduce the concentration of the active ingredient on the surface of the particles. Such coating may be performed in order to partially or fully encapsulate the active ingredient on the surface of the particle, thereby reducing burst release of the active ingredient.

### Compositions

In one embodiment, the particles are dispersed in a matrix comprising (i) a polymer which is not a CYC carrier (which can be the same as or different from any non-CYC polymer which is present in the particles) and/or (ii) a CYC carrier, which can be the same as or different from the CYC carrier in the particles. The second matrix can for example comprise a CYSC acrylate or other CYSC polymer emulsion or another film-forming polymer, for example as described in the documents incorporated by reference herein. The second matrix optionally comprises a release material, which may be the same as or different from a release material in the particles. Like the first matrix, the second matrix can include other materials, for example, fillers, surface active agents etc.

In some embodiments, the compositions of the invention are solid, e.g. are in the form of a dry coating on a substrate, for example a seed. In other embodiments, the compositions comprise a liquid carrier, for example so that the composition is in a form suitable for application to a seed or other substrate to form a coating on the substrate. By appropriate selection of the matrix materials, the active ingredients and the ambient conditions, these compositions will deliver the active ingredients at a desired rate. The active ingredient can pass through the amorphous domains much more easily than it can pass through the crystalline domains (through which the rate of passage may be negligible). The rate of release can be increased by heating the composition to a temperature above Tp. However, in many cases, the compositions of the invention are used at temperatures which do not include the melting range of the semicrystalline polymer. Under these circumstances, the rate of release depends primarily upon the dimensions of the tortuous paths in the microstructure, the nature of the active ingredient, and the access of water or other displacing medium to the microstructure. When the particles are dispersed in a second matrix, the second matrix can also be important in determining the rate of release. The same or different release materials can be released at the same or different rates and/or at different times. In this way, for example, the active ingredient can be released slowly or not at all initially, and be released more rapidly when delivery of it is desirable.

The particles can also be dispersed into a preformed composition, optionally a composition as disclosed in any of the documents incorporated by reference herein, e.g. a drug formulation, a personal care composition (e.g. a hand lotion, shampoo or sun care composition) or a seed coating composition.

### EXAMPLES

The invention is illustrated by the following Examples.

The CYSC polymers used in the Examples were prepared by polymerizing the monomers, and the amounts of the monomers in parts by weight, shown in Table 1. The following abbreviations are used in Table 1. AA is acrylic acid semitone C16A hexadecyl acrylate; C22A is behenyl acrylate; and 2-HEA is 2-ethyl hexyl acrylate. The polymers were prepared by solution polymerization of the monomers in isopropyl alcohol at about 80° C. for 3 hours under a nitrogen blanket in the presence of about 0.1% AIBN, and in some cases about 6% by weight of butyl 3-mercapto propionate to control the molecular weight. At the end of this reduced pressure stage, the internal temperature was about 120-130° C. and 0.5 g of tert-butyl peroxy-3,5,5-trimethylhexanoate (Triganox 42S) was added to reduce the residual monomer concentration. The reaction was continued at 120-130° C. for at least 1 hour, followed by 1 hour under reduced pressure. Table 1 also shows the weight average molecular weight, viscosity and Tp of the polymers.

Table 2 shows the ingredients of the samples which were tested, and the amounts of the ingredients in parts by weight. The following abbreviations are used in Table 2. SA-2 is (polyoxyethylene)₂ stearyl alcohol; SA-6 is (polyoxyethylene)₆stearyl alcohol; and IMI is imidacloprid. Table 2 also shows the method used to prepare the samples and the particle size (PSA) of the samples. In method 1, the ingredients were processed on a spinning disk at a temperature of 110-120° C. (which is above the Tp of the polymer, but below the melting point of the IMI) at a disk speed of 10,000 RPM. In method 2, the ingredients were processed at a temperature of 165-182° C. (which is above the Tp of the polymer, and above the melting point of the IMI) at a disk speed of 25,000 RPM. The particle size of the product was measured by dispersing 20-50 mg of the particles in 3 mL of DI water containing 2% Tween 20, followed by agitation using a vortex mixer to break up agglomerated particles. The sample was added to the sample reservoir in a Horiba LA-910 Laser Scattering Particle Size Distribution Analyzer. The first data point was measured without sonication. The sample was then sonicated in the reservoir for 1 minute, and a second data point was measured. The results are reported as volume weighted average after the first sonication.

TABLE 1

| Polymer # | C16A | C22A | HEA | | Mw | Viscosity | Tp |
|---|---|---|---|---|---|---|---|
| 351-192-1 | — | 82 | 18 | — | 21,292 | 430 | 66 |
| I0 209 | — | 95 | — | 5 | 50,072 | 140 | 66 |
| 351-175-1 | 90 | — | — | 10 | 8093 | 585 | 34 |
| H4 CO 815 | — | 82 | 18 | — | 24,000 | 590 | 66 |

TABLE 2

| | | Polymer | | IMI | SA-2 | SA-6 | | |
|---|---|---|---|---|---|---|---|---|
| Ex# | Sample | # | Wt | Wt | Wt | Wt | Method | PSA |
| t | p 03 | 10209 | 65 | 35 | — | — | 1 | 94 |
| 2 | p 05 | 10209 | 49.4 | 34 | 16.6 | — | 1 | 69 |
| 3 | p 06-1 | 10209 | 49.4 | 34 | 16.6 | — | 1 | 90 |
| 4 | p 06-2 | 10209 | 65 | 30 | — | 5 | 1 | 94 |
| 5 | p 06-9 | H4CO815 | 70 | 30 | — | — | 1 | 144 |
| 6 | p 08-9 | H4CO815 | 70 | 30 | — | — | 2 | 111 |
| 7 | p 09-13 | 351-175-1 | 70 | 30 | — | — | 1 | 118 |
| 8 | p 09-14 | 351-175-1 | 60 | 30 | 10 | — | 1 | 110 |
| 9 | p 09-15 | 351-175-1 | 65 | 30 | 10 | — | 1 | 116 |
| 10 | p 12-22 | 351-192-1 | 70 | 30 | — | — | 2 | 83 |
| 11 | p 12-23 | 351-192-1 | 65 | 30 | 5 | — | 2 | 85 |
| 12 | p 17-32 | 351-192-1 | 65 | 30 | — | 5 | 2 | ≦50 |
| 13 | p 17-30 | H4CO815 | 65 | 30 | — | 5 | 2 | 108 |
| 14 | p 17- 31 | H4CO815 | 65 | 30 | — | 5 | 2 | 29 |

### Testing

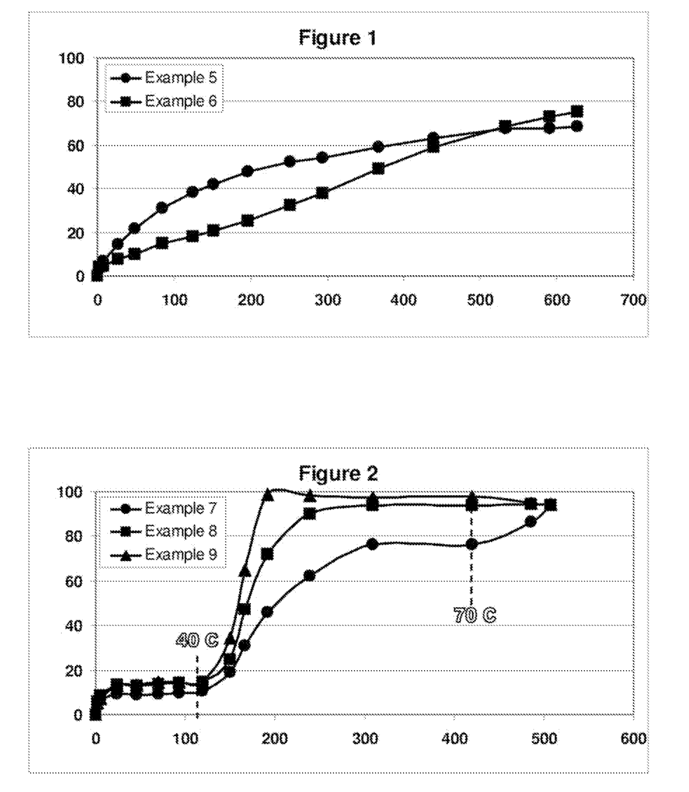

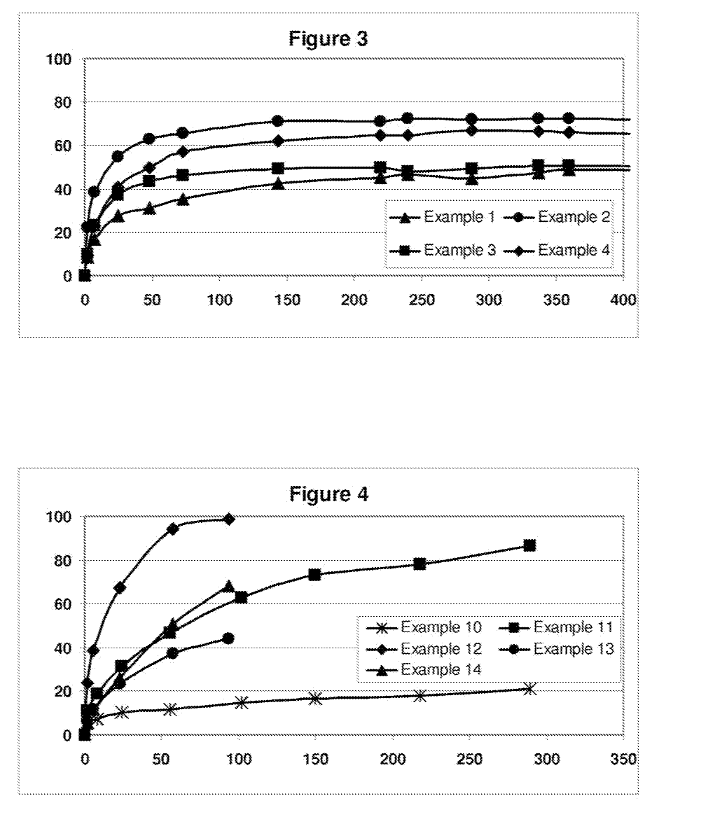

The microparticles (15-20 mg), and water (20 g) were shaken together in a 20 mL vial. 100 μl aliquots were removed periodically, diluted to 3 g with water, and assayed at 269 nm (using a Perkin Elmer spectrophotometer) to determine the amount of imidacloprid released. The results are shown in FIGS. 1-4. In FIGS. 1 and 3, the entire test was conducted at 30° C. In FIG. 2, the test was conducted at 30° C. for the first 150 hours, at 40° C. (which is above the Tp of the polymer in the samples tested in FIG. 2) for another 270 hours, and thereafter at 70° C. In FIG. 4, the entire test was conducted at 25° C.

1. A composition which comprises a multiplicity of particles, each of the particles comprising (1) a microstructure comprising a semicrystalline polymer which has a peak melting temperature, Tp, of at least 20° C., measured as hereinbefore defined, and (2) an active ingredient which is a release material as hereinbefore defined;
the microstructure comprising
crystalline domains of the semicrystalline polymer, the crystalline domains being separate from each other, and
(ii) amorphous domains of the semicrystalline polymer, the amorphous domains providing a multiplicity of tortuous paths between the crystalline domains; and
at least part of the active ingredient being present in the amorphous domains.

2. A composition according to claim 1 wherein the microstructure has a characteristic length scale between 5 nm and 1000 nm, measured by small-angle X-ray scattering as hereinbefore defined.

3. A composition according to claim 1 or 2 wherein the semicrystalline polymer is a CYC carrier as hereinbefore defined.

4. A composition according to claim 3 wherein the semicrystalline polymer is a CYSC polymer as hereinbefore defined, the CYSC polymer having at least one of the following characteristics
(i) a heat of fusion of at least 20, preferably at least 30, particularly at least 40, J/g, measured as hereinbefore before defined,
(ii) a peak melting temperature, Tp, and an onset of melting temperature, To, such that (Tp−To) is less than Tp^{0.7}, preferably less than Tp^{0.6}, particularly less than 10° C., measured as hereinbefore defined,
(iii) a peak melting temperature, Tp, of at least 30° C., e.g. at least 35° C., for example 35-80° C., or 50-65° C., or at least 50° C., e.g. 15-85° C. measured as hereinbefore defined,
(iv) a number average molecular weight of 1500-100,000, e.g. 5000-60,000, 5000-50,000, or 5000-30,000, for example 10,000-20,000, or 15,000-25,000, and/or a weight average molecular weight of 2000-100,000, e.g. 10,000-100,000, preferably 15,000-25,000,
(v) it contains repeating units containing functional groups, for example units derived from monomers containing one or more oxygen-containing, nitrogen-containing, or silicon-containing groups, e.g. carboxyl, hydroxyl, amino, substituted amino, amido or polyoxyethylene groups, the polymer containing for example at least 5%, at least 10%, at least 15%, at least 30%, or at least 40%, in some cases 30-50%, by weight of such units, and
(vi) it has a melt viscosity less than 1000 cps, for example less than 500 cps measured as hereinbefore defined, and
(vii) it is a block copolymer.

5. A composition according to any one of the preceding claims which contains an additive which is mixed with the semicrystalline polymer, and has at least one of the following characteristics:—
(a) it decreases the Tg of the polymer,
(b) it increases the Tg of the polymer,
(c) it increases the Tp of the polymer,
(d) it increases the solubility of the active ingredient in the polymer,
(e) it modifies the active ingredient or the polymer to improve compatibility of the active ingredient and the amorphous domains of the polymer,
(f) it modifies the surface properties of the active ingredient or the polymer to improve compatibility of the active ingredient and the crystalline domains of the polymer,
(g) it provides additional crystalline domains, and
(h) it provides additional amorphous domains.

6. A composition according to claim 5 wherein the additive
(a) is water, a solvent, a plasticizer or a low melting solid having a melting point less than (Tp−10) ° C., or
(b) is a crystalline alcohol having a melting point less than Tp, for example neopentyl glycol, tertiary butyl alcohol, or cyclohexylamine dimethanol, or
(c) comprises hydrophobic groups, e.g. long chain alkyl groups, which associate with side chains of a CYSC polymer, for example, is an alcohol containing a straight chain alkyl group containing at least 14, preferably at least 16, e.g. 16-24, carbon atoms, or an ester of an acid and such an alcohol, or.
(d) comprises amphiphilic moieties, e.g. ethoxylates of long chain alcohols preferably containing at least 16, e.g. 16-24, carbon atoms, for example containing up to 20 ethoxylates groups, preferably 3-6 ethoxylate groups, or
(e) is a low molecular weight polymer.

7. A composition according to any one of the preceding claims wherein substantially all of the release material is dispersed in the amorphous domains of the semicrystalline polymer.

8. A composition according to any one of claims 1-6 wherein part of the release material is present in domains separate from the crystalline and amorphous domains of the semicrystalline polymer

9. A composition according to any one of the preceding claims wherein the particles have an average particle size of 0.5-250μ, e.g. 0.500 150, 10-150, 21-150:20-70, or 0.5-25, micron.

10. A composition according to any one of the preceding claims wherein the particles are dispersed in substantially continuous matrix which is a liquid or a solid, preferably a solid polymer, the matrix optionally comprising a CYC carrier.

11. A composition according to any one of the preceding claims which is in the form of a coating on a seed.

12. A method of making a composition as claimed in any one of the preceding claims which comprises.
(A) dispersing the release material in the semicrystalline polymer; and
(B) converting the dispersion prepared in step (A) into the particles; wherein step (A) comprises
(A1) homogenizing a mixture of the polymer and the active ingredient at a temperature above the Tp of the polymer and below the melting point of the active ingredient, or
(A 2) homogenizing a mixture of the polymer and the active ingredient at a temperature above the Tp of the polymer and above the melting point of the active ingredient, and
step (B) comprises rapidly cooling the dispersion prepared in step (A) to a temperature substantially below Tp.

13. A method according to claim 12 wherein step (B) comprises treating the dispersion prepared in step (A) on a spinning disk apparatus.

14. A method according to claim 12 or 13 wherein step (B) comprises cooling the dispersion to a temperature below 10° C., and grinding the cooled dispersion.

15. A method of delivering an active ingredient which comprises exposing a composition as claimed in any one of claims 1-11 to water or another liquid which is soluble in the amorphous domains, while the composition is at a temperature below Tp and preferably while the composition is at a temperature above Tg.

16. A composition which comprises a multiplicity of particles, each of the particles comprising (1) a CYC carrier as hereinbefore defined, and (2) a release material which is an active ingredient as hereinbefore defined, and which is associated with CYC carrier, each of the particles comprising a microstructure comprising
(iii) first domains, preferably crystalline domains, which are separate from each other, and
(iv) second domains, preferably amorphous domains, which provide a multiplicity of tortuous paths between the first domains,
the release material (a) contacting the second domains, (b) having a comparatively low rate of diffusion through the first domains, and (c) having a comparatively high rate of diffusion through the second domains.
